(19) United States
(12) Patent Application Publication
(10) Pub. No.: US 2012/0197194 A1
(43) Pub. Date: Aug. 2, 2012

(54) Folding balloon catheter

(75) Inventor: Thomas P. Osypka, Palm Harbor, FL (US)

(73) Assignee: Oscor Inc., Palm Harbor, FL (US)

(21) Appl. No.: 13/343,228

(22) Filed: Jan. 4, 2012

Related U.S. Application Data
(60) Provisional application No. 61/460,525, filed on Jan. 4, 2011.

(51) Int. Cl. A61M 25/10

(52) U.S. Cl. 604/103.07

(57) ABSTRACT

A catheter assembly including a tubular assembly consisting of an inner tubular member slidably received within an outer tubular member such that a distal end portion of the inner tubular member extends distally from a distal end of the outer tubular member. The catheter assembly further includes a balloon member having first and second end regions wherein a first end region is attached to the distal end portion of the inner tubular member and the second end region is attached to the distal end portion of the outer tubular member.

### CROSS REFERENCE TO RELATED APPLICATIONS

This application claims priority to U.S. patent application Ser. No. 61/460,525 filed Jan. 4, 2011 which is incorporated herein by reference in its entirety.

### FIELD OF THE INVENTION

The present invention generally relates to balloon catheters. More particularly, the present invention relates to a foldable medical balloon catheter device and process of manufacture for providing a balloon catheter that folds onto a catheter tube during balloon deflation.

### BACKGROUND OF THE INVENTION

A balloon catheter generally comprises an inflatable balloon that is mounted along a distal end of an elongate catheter body (i.e., shaft). Balloon catheters are used by physicians in a wide variety of therapeutic procedures. In one common use, a folded balloon catheter is advanced through a blood vessel to a region that has become occluded by atherosclerotic plaque. The balloon is inflated to dilate the occluded region and thereby improve the flow of blood through the vessel. In another common use, an expandable stent is provided along the exterior of the balloon. The balloon is advanced to the treatment site and is then inflated to deploy the stent. The balloon is then deflated and the balloon catheter is withdrawn from the patient. The expanded stent remains in the blood vessel to provide support to the vessel wall.

Balloon catheters are typically formed of a very thin, yet strong material. During manufacture, a balloon is folded at a number of locations along its longitudinal axis. After the balloon is folded in a variety of locations, the folds are wrapped around the catheter to reduce the balloon to a constrained condition having a very small diameter. In the case wherein the balloon is used to deploy a stent, the stent is crimped onto the balloon after the folding step. The balloon is advanced through the blood vessel to a treatment site while in the constrained condition. The balloon is typically inflated by directing a fluid through a lumen in the catheter to pressurize the balloon. During inflation, the balloon unfolds, rather than stretches.

It is to be appreciated that arterial blockages are typically caused by the buildup of plaque in the arteries of a patient often having severe consequences. This is because the buildup of plaque in arteries reduces, and eventually, blocks blood flow through the affected vessel. When blood flow is reduced in a coronary artery, the heart muscle becomes deprived of oxygen, and the patient is prone to suffer angina. In severe cases of coronary artery blockage, the patient suffers a heart attack.

Many modern surgical techniques have been developed to alleviate the stenoses that are formed when plaque builds up in a patient's arteries. For example, a large number of balloon angioplasty devices exist for relieving arterial stenoses by compression of the stenosis. In several respects, balloon angioplasty devices afford numerous advantages over alternative methods. Foremost among these advantages is that open heart bypass surgery can often be avoided by using angioplasty surgical techniques to relieve stenoses in the arteries that supply blood to the heart. For obvious reasons, it is preferable to avoid open heart surgery when possible because such surgery, as is well known, is invasive and typically requires a significant post-operative recovery time. Accordingly, it is preferable to use relatively simpler angioplasty surgical procedures when such procedures are feasible. Importantly, angioplasty procedures are efficacious in the peripheral arteries as well as in the arteries that supply blood to the heart.

In angioplasty surgery, the balloon of a balloon catheter is initially attached to a catheter tube in a deflated configuration, with the catheter tube connecting a fluid source in fluid communication with the balloon. The balloon is then positioned at the desired location in the affected artery by advancing the catheter through the artery until the balloon is positioned across a stenosis that is to be treated. Once the balloon has been properly positioned, fluid is infused into the balloon. As the balloon expands, it dilates the lumen of the artery and compresses the plaque which may then break up or flatten out against the arterial wall. The balloon is then deflated and, once in its deflated configuration, it is either withdrawn from the artery or placed across another stenosis, to restore normal blood flow through the artery.

However, a noted problem associated with an angioplasty procedure exists during the deflation stage of the balloon, prior to its removal from the artery. Specifically, it is desirable that the balloon be deflated as tightly as practicable to facilitate its removal from the arterial passageways. It is desirable when removing a balloon catheter to have the balloon collapse evenly and compactly during balloon deflation. This is because, once deflated, the balloon catheter must often travel through the tortuous vasculature of the patient and it is, therefore, important for the balloon to deflate uniformly into a predictable configuration. If the balloon fails to deflate in a uniform manner, an irregular bulge or even sharp edge in the balloon may cause difficulties in withdrawing the balloon catheter from the artery. In particular when non compliant high pressure balloons are deflated they tend to fold upon themselves in flat leaf-like shape, having sharp edges on the side, which makes the retraction of the balloon into the guiding or introducer sheath cumbersome or sometimes even impossible, requiring removal of the complete system including surgical removal.

Accordingly, it is an object of the present invention to provide a balloon catheter having a mechanism, which causes the balloon to consistently fold and wrap itself up predictably, compactly and tightly onto a catheter tube during balloon deflation to facilitate in vivo movement of the balloon catheter in a uniform matter.

Another object of the present invention is to provide a device which is relatively simple to manufacture, easy to use, and comparatively cost effective.

### SUMMARY OF THE INVENTION

Embodiments of the present invention disclosed herein provide improved devices and methods for folding and wrapping balloon catheters and to enable a balloon catheter to radially expand and contract in a very predictable manner. The preferred embodiments facilitate the manufacture of balloon catheters while minimizing costs and providing a balloon catheter that expands and contracts in a reliable and predictable manner. Further, the preferred embodiments provide a modular unit that is capable of folding and wrapping balloon catheters of a variety of different sizes and shapes.

### BRIEF DESCRIPTION OF THE DRAWINGS

The objects and features of the invention can be understood with reference to the following detailed description of an illustrative embodiment of the present invention taken together in conjunction with the accompanying drawings in which:

FIGS. 1 to 5 illustrate the process for forming a foldable balloon catheter in accordance with the present invention; and

FIGS. 6 to 13 illustrate a method of use for the present invention foldable balloon catheter.

### WRITTEN DESCRIPTION OF CERTAIN EMBODIMENTS OF THE INVENTION

The present invention is now described more fully with reference to the accompanying drawings, in which an illustrated embodiment of the present invention is shown. The present invention is not limited in any way to the illustrated embodiment as the illustrated embodiment described below is merely exemplary of the invention, which can be embodied in various forms, as appreciated by one skilled in the art. Therefore, it is to be understood that any structural and functional details disclosed herein are not to be interpreted as limiting, but merely as a basis for the claims and as a representative for teaching one skilled in the art to variously employ the present invention. Furthermore, the terms and phrases used herein are not intended to be limiting but rather to provide an understandable description of the invention.

It is to be appreciated and understood that the present invention, in accordance with the illustrated embodiments, is directed to a system and process for assembling balloons onto the catheter shaft such that the balloon is folded onto itself tightly around the catheter in a twisted (overlapping) matter each time the balloon is caused to be deflated.

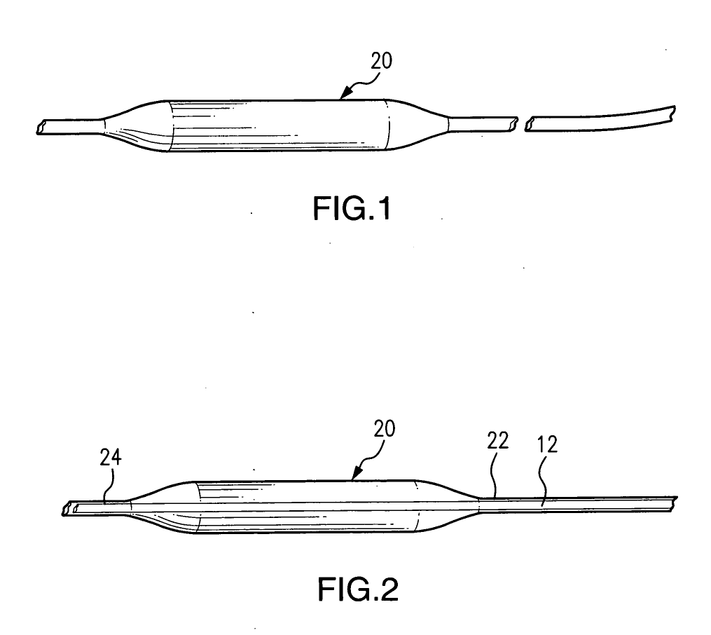

As will be apparent from the below description in accordance with the illustrated embodiments of FIGS. 1 to 13, the balloon catheter 10 in accordance with the illustrated embodiments of the invention includes an outer shaft tubing 12 and a distal inner shaft tubing 14 (e.g., a guidewire lumen—as the inner shaft lumen is often used as a guidewire lumen, whereby the balloon catheter is introduced over a guidewire into the vessel to the desired location) and a balloon 20 affixed thereto as described below. It is to be appreciated the inner shaft tubing 14 resides the length of the catheter 10 and is slideably received within the outer tubing 12 having a distal end portion 16 extending distally from the distal end 18 of the outer tubing 12. A proximal section 22 of a balloon 20 is mounted onto the outer tubing 12 by known adhesive means (e.g., heat, glue, and the like) with a distal section 24 of the balloon 20 being mounted onto the inner tubing 14 also by known adhesive means (e.g., heat, glue, and the like) such that the balloon 20 is sealed between both the inner 14 and outer tubing 12 members in an manner suitable for inflation.

Further, a self twisting and self wrapping of the balloon 20 during inflation and deflation is achieved whereby the distal end 24 of the balloon 20 is prescribed with an at least 45° twisted turn orientation relative to the proximal section 22 of the balloon 20 which is then attached to the distal inner tubing 14 in a twisted condition such that the inner tubing 14 achieves a constant torque of the balloon 20 to twist and fold itself. Depending on the shape and size of the balloon, several twists exceeding 360° may be provided.

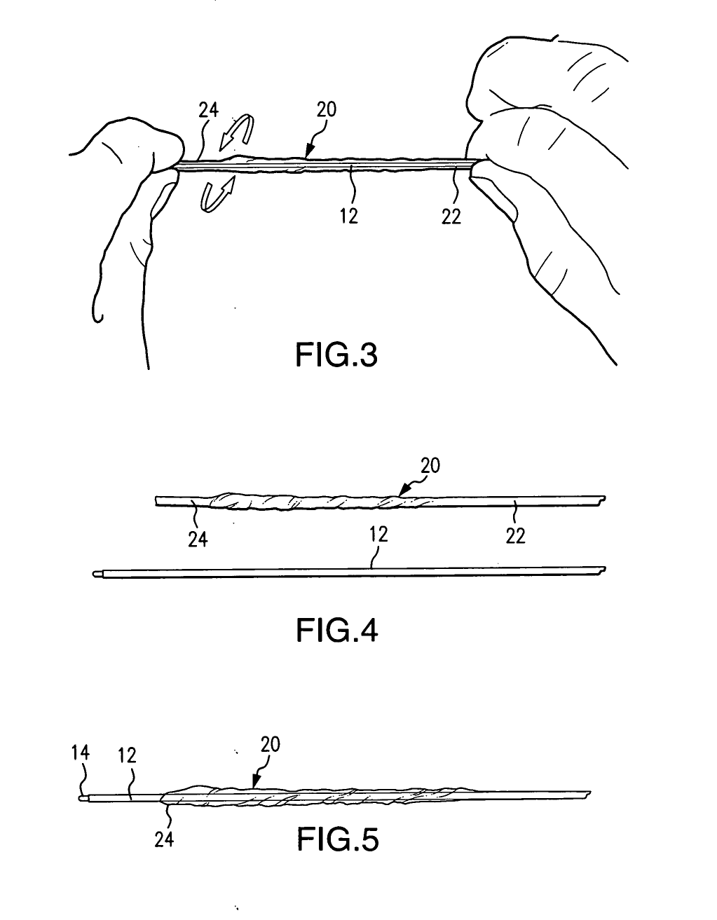

Turning now descriptively to the drawings, in which similar reference characters denote similar elements throughout the several views, FIG. 1 depicts a balloon 20 for use with a catheter device 10. Preferably medical grade material is used to fabricate balloon 20 which material is typically very thin but rather rigid plastic, so that the inflated diameter is predictable and doesn't vary greatly as a function of inflation pressure. Because of this, catheter balloons do not stretch like a rubber balloon when inflated, but rather they unfold. FIG. 2 depicts an outer shaft tubing member 12 (having a inner shaft tubing member 14 slidably received therewithin) received through the interior portion of the balloon 20 so as to extend from the proximal section 22 of balloon 20 to its distal end section 24. FIG. 3 depicts the folding of balloon 20 about outer shaft tubing 12. It is to be appreciated folding is the process of wrapping the wing portions of balloon 20 spirally around the outer shaft tubing 12. Folding may be done by either hand or machine. When performed by hand as shown in FIG. 3, it is accomplished by holding the shaft 12 in one hand while gripping and turning the adjacent part of the balloon 20 around the catheter axis with the other hand. The balloon 20 is preferably folded incrementally, moving both the folding and grasping hands incrementally in steps from the distal 24 to the proximal 22 end of the balloon 20. Following the folding, the balloon 20 is preferably placed in an introducer sheath to hold it in the folded position and guide it to a desired surgical location. FIG. 4 depicts the balloon 20 in a folded position on outer shaft tubing 12 ready for insertion into the lumen 102 of an introducer 100 while FIG. 5 depicts the balloon 20 in at least a partially inflated position.

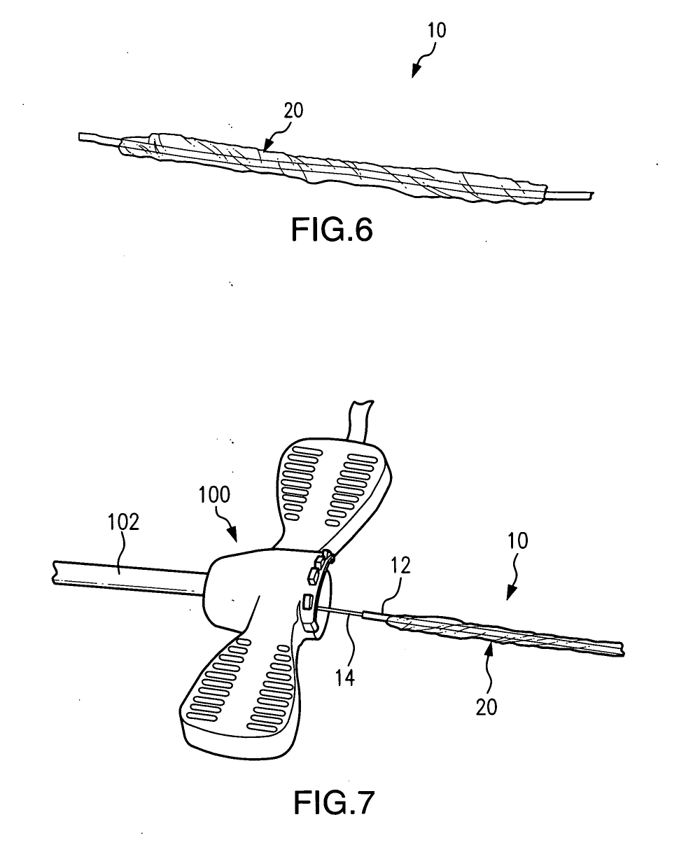

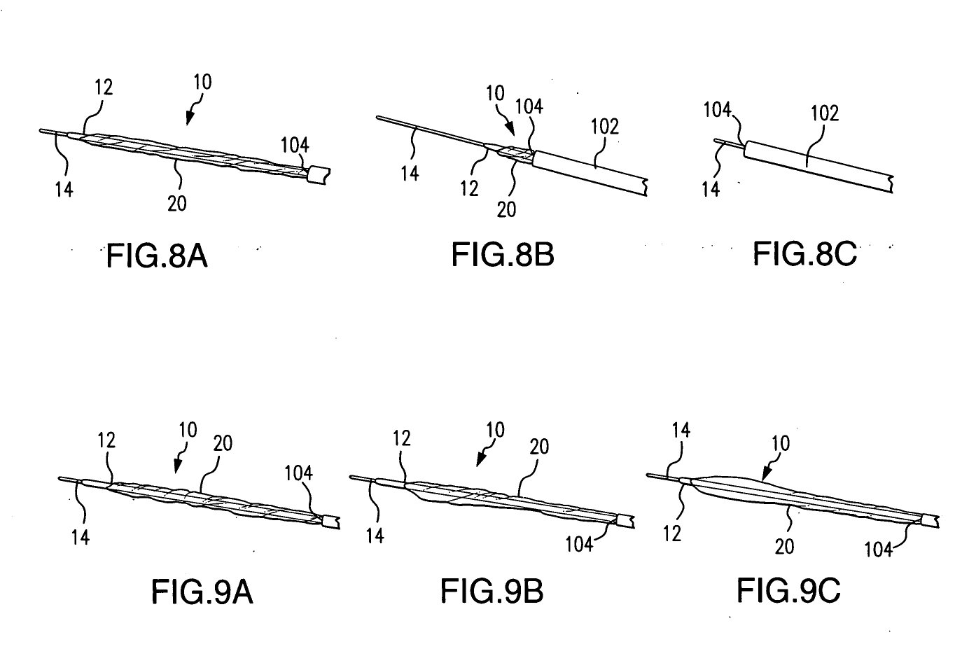

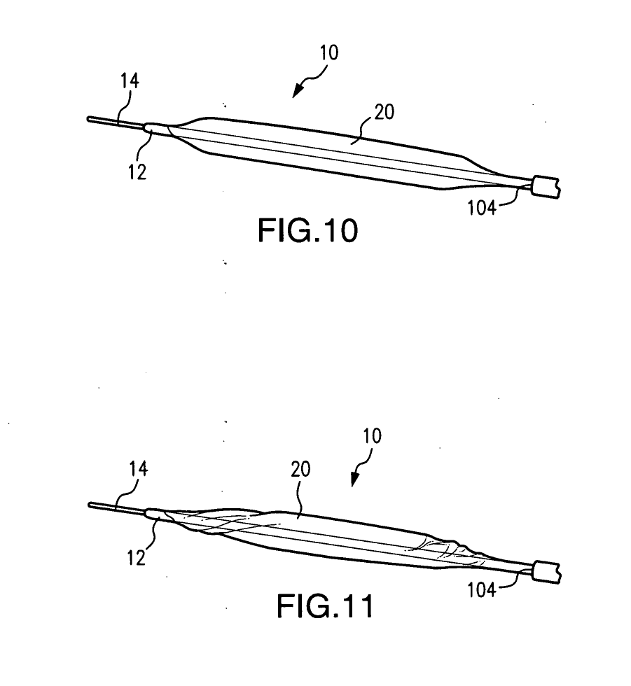

Reference is now made to FIGS. 6 to 13 illustrating the self-twisting balloon features of the present invention. With reference to FIG. 6, illustrated is a balloon 20 of balloon catheter 10 in a fully wrapped position, preferably in preparation for insertion into the lumen 102 of an introducer sheath 100. FIG. 7 illustrates the balloon catheter 10 (in which the balloon 20 is tightly wrapped around the outer shaft tubing 12 of catheter 10) being introduced into the lumen 102 of introducer sheath 100. FIGS. 8A to 8C illustrate the sequential steps of the balloon catheter 10 (of FIG. 6) being slideably received within the lumen 102 of introducer sheath 100 such that it extends distally from the distal end 104 of the lumen 102 (which is to be understood to be inserted in a vessel) of introducer 100 so as to be exposed to a vessel (FIG. 8C). FIGS. 9A to 9C illustrate the sequential steps of balloon 20 being inflated (via preferably an inflation fluid, such as saline solution or a contrast medium) such that the portion of the balloon 20 extending from the distal end 104 of the lumen sheath 102, unwraps (e.g., unfolds) from the outer shaft tubing 12 of catheter 10. FIG. 10. depicts the balloon 20 in a fully inflated position while FIG. 11 depicts the balloon 20 being deflated, preferably via the use of a pulling vacuum provided on an inflation syringe wherein it is to be appreciated and understood, in accordance with the present invention, the balloon wraps (folds) itself into the wrapped position shown in FIGS. 3A to 3C.

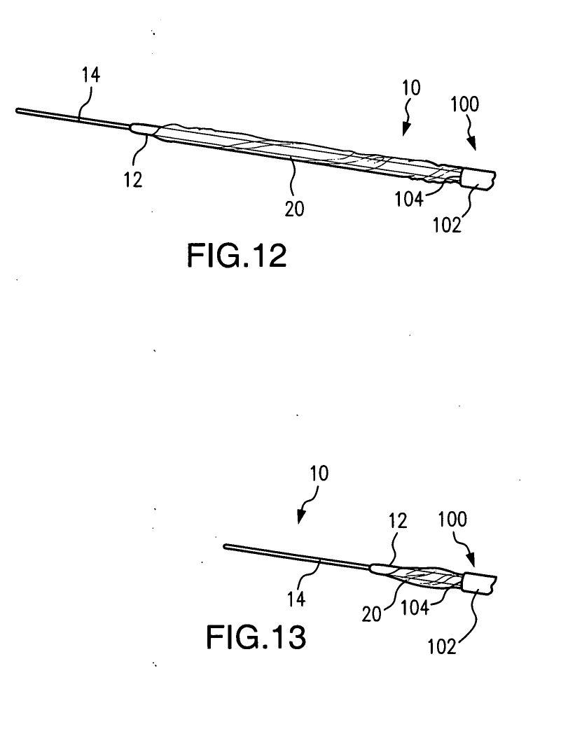

With reference to FIG. 12, illustrated is the balloon 20 in a fully deflated and tightly wrapped position about the outer shaft tubing 12 of catheter 10. As illustrated in FIG. 13, this enables the balloon catheter 12 to be retracted back into the lumen 102 of introducer 100 such that the balloon catheter 10 may be transported to another surgical location or removed from the patient's body via introducer 100.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

The above presents a description of a best mode contemplated for carrying out the present balloon folding and wrapping devices and methods, and of the manner and process of making and using them, in such full, clear, concise, and exact terms as to enable any person skilled in the art to which it pertains to make and use these devices and methods. These balloon folding and wrapping devices and methods are, however, susceptible to modifications and alternative method steps from those discussed above that are fully equivalent. Consequently, these balloon folding and wrapping devices and methods are not limited to the particular embodiments disclosed. On the contrary, these balloon folding and wrapping devices and methods cover all modifications and alternative constructions and methods coming within the spirit and scope of the present invention. For instance, a device using a single outer tubing and employing two discrete lumens may also be used in addition to what is illustrated.

The descriptions above and the accompanying drawings should be interpreted in the illustrative and not the limited sense. While the invention has been disclosed in connection with the preferred embodiment or embodiments thereof, it should be understood that there may be other embodiments which fall within the scope of the invention as defined by the following claims. Where a claim, if any, is expressed as a means or step for performing a specified function, it is intended that such claim be construed to cover the corresponding structure, material, or acts described in the specification and equivalents thereof, including both structural equivalents and equivalent structures, material-based equivalents and equivalent materials, and act-based equivalents and equivalent acts.

What is claimed is:

1. A balloon catheter comprising:
a first outer tubular member;
a second inner tubular member configured to a slideably receive within the first outer tubular member such that a distal end of the second inner tubular member extends distally from a distal end of the first outer tubular member; and
an inflatable balloon having opposing distal and proximal ends wherein the proximal end portion of the balloon is affixed to the distal end portion of the first outer tubular member and the distal end portion of the balloon is affixed to the distal end portion of the second inner tubular member wherein the balloon resides on the first and second tubular members with an at least 45° or more twisted turn orientation relative to the proximal section of the balloon attached to the first outer tubular member such that the second inner tubing achieves a constant torque of the balloon to twist and fold itself when retracted back into a portion of the first outer tubular member.

2. A balloon catheter as recited in claim 1 wherein the balloon is fabricated with rigid plastic.

3. A balloon catheter as recited in claim 1 wherein the balloon catheter is configured and adapted to be inserted within an introducer sheet lumen.

4. A balloon catheter as recited in claim 1 wherein the balloon is configured and adapted to be inflated with liquid.

5. A balloon catheter as recited in claim 1 wherein the balloon is configured and adapted to return to it's twisted turn orientation relative to the outer tubular member when the balloon is caused to be deflated.

6. A catheter assembly comprising:
a tubular assembly consisting of an inner tubular member slidably received within an outer tubular member such that a distal end portion of the inner tubular member extends distally from a distal end of the outer tubular member; and
a balloon member having first and second end regions wherein a first end region is attached to the distal end portion of the inner tubular member and the second end region is attached to the distal end portion of the outer tubular member.

7. A catheter assembly as recited in claim 6 wherein the balloon resides on the tubular assembly with at least 45° or more twisted turn orientation relative to the second end region of the balloon attached to the distal end portion of the outer tubular member.

8. A catheter assembly as recited in claim 7 wherein the inner tubular member achieves a constant torque of the balloon to twist and fold itself when retracted back into a portion of the outer tubular member.

9. A balloon catheter as recited in claim 6 wherein the balloon is fabricated with rigid plastic.

10. A balloon catheter as recited in claim 6 wherein the tubular assembly is configured and adapted to be inserted within an introducer sheet lumen.

11. A balloon catheter as recited in claim 6 wherein the balloon is configured and adapted to be inflated with liquid.

12. A balloon catheter as recited in claim 6 wherein the balloon is configured and adapted to return to it's twisted turn orientation relative to the outer tubular member when the balloon is caused to be deflated.

13. A method of inflating a balloon catheter at a surgical site, the method comprising the steps of:
providing an introducer sheet defining an inner lumen;
providing a tubular assembly consisting of an inner tubular member slidably received within an outer tubular member such that a distal end portion of the inner tubular member extends distally from a distal end of the outer tubular member wherein a balloon member is attached to the distal end portion of the inner tubular member at first end region and a second end region of the balloon member is attached to the distal end portion of the outer tubular member;
slideably receiving the tubular assembly within the inner lumen of the introducer sheet;
positioning the introducer sheet at the surgical site;
advancing the tubular assembly from a distal end of the introducer sheet such that the balloon member is exposed from the inner lumen of the introducer sheet;
inflating the balloon member at the surgical site;
deflating the balloon member at the surgical site;
retracting the tubular assembly into the inner lumen of the introducer at the distal end of the introducer sheet such that a substantial portion of the balloon member is located within the inner lumen of the introducer sheet.

14. A method of inflating a balloon catheter at a surgical site as recited in claim 14 wherein the balloon resides on the tubular assembly with at least 45° or more twisted turn orientation relative to the second end region of the balloon attached to the distal end portion of the outer tubular member.

15. A method of inflating a balloon catheter at a surgical site as recited in claim 14 wherein when the balloon is caused to inflate, it un-twists itself relative to the distal end portion of the outer tubular member.

16. A method of inflating a balloon catheter at a surgical site as recited in claim 15 wherein when the balloon is caused to deflate, it returns to its said twisted orientation relative to the distal end portion of the outer tubular member when retracted back into a portion of the outer tubular member.

17. A method of inflating a balloon catheter at a surgical site as recited in claim 16 wherein the inner tubular member achieves a constant torque of the balloon to twist and fold itself when retracted back into a portion of the outer tubular member.

18. A method of inflating a balloon catheter at a surgical site as recited in claim 14 wherein the balloon is fabricated with rigid plastic.

19. A method of inflating a balloon catheter at a surgical site as recited in claim 14 wherein the balloon is configured and adapted to be inflated with liquid.
